(19) United States
(12) Patent Application Publication
(10) Pub. No.: US 2022/0348862 A9
(43) Pub. Date: Nov. 3, 2022

(54) SYSTEM AND METHOD FOR OPTIMIZATION OF THE FERMENTATION PROCESS

(71) Applicant: Buckman Laboratories International, Inc., Memphis, TN (US)

(72) Inventors: Bret Magness, Collierville, TN (US); João Ducatti, Campinas (BR); Erika Balzuweit Lopes, Ribeirao Preto (BR); Egnaldo Samento dos Santos, Campinas (BR); John Kurtz, Memphis, TN (US); Carolina Mendes Morgante, Vinhedo (BR); Rafael Lopes Duarte Barros, Rio de Janeiro (BR); Dave Howard, Memphis, TN (US); Amit Sharma, Memphis (IN); Nate Brandeburg, Memphis, TN (US); Dimitri Kuznetsov, Memphis, TN (US)

(21) Appl. No.: 16/927,588

(22) Filed: Jul. 13, 2020

Related U.S. Application Data
(60) Provisional application No. 62/873,831, filed on Jul. 12, 2019.
(60) Provisional application No. 62/880,522, filed on Jul. 30, 2019.
(60) Provisional application No. 63/001,975, filed on Mar. 30, 2020.

(51) Int. Cl. C12M 1/34; B01D 19/00; B01D 19/04; C12M 1/00; C12M 1/36

(52) U.S. Cl. CPC C12M 41/44; B01D 19/0036; B01D 19/0063; B01D 19/04; C12M 29/20; C12M 23/58; C12M 41/48

(57) ABSTRACT

The invention comprises one or more gas volume fraction measurement devices operatively connected to one or more controllers and one or more deaeration mechanisms which receive control signals from said one or more controllers and perform an act on the system, such as by controlling a level of deaeration chemistry into some portion of the fermentation system. In one embodiment, the deaeration mechanism is an antifoam feed pump which pumps antifoam chemistry into a feed line of the fermenter in response to the measured gas volume fraction in the fermenter's recirculation loop, in an amount determined by the controller to be effective to reduce foaming and lower column height in the fermenter.

### CROSS-REFERENCE TO RELATED APPLICATIONS

The present invention claims priority to U.S. Provisional Patent Application No. 62/873,831, filed Jul. 12, 2019, U.S. Provisional Patent Application No. 62/880,522, filed Jul. 30, 2019, and U.S. Provisional Patent Application No. 63/001,975, filed Mar. 30, 2020, all of which are incorporated herein by reference in their entireties.

### BACKGROUND OF THE INVENTION

### Field of the Invention

The invention relates primarily to solutions for systems and methods for actively controlling gas volume fraction in a fermentation vessel. More specifically, the present invention is a novel system, and methods for using same, which provides proactive, real-time control over various processing parameters in a fermenter to reduce foam in the fermenter vessel.

### Description of the Background

Sugarcane juice is one of the raw materials used in the production of ethanol through the biochemical process of fermentation. The biochemical process of fermentation starts with the alimentation of the sugar-containing juice and yeast to tanks known as fermentation tanks. The reaction process generates, as major products, ethanol and carbon dioxide (CO₂) in equal parts and in quantities that vary based on different process variables. As long as sugar remains in the reaction liquid, however, yeast will continue to consume this sugar to produce ethanol and CO₂. The reaction process is exothermic, generating heat which must be removed.

The carbon dioxide generated by the yeast inherently affects the fermentation process by decreasing the tank working volume through foam creation and its turbulent release inside the liquid. This entrained gas and resulting foam generation creates difficulties in maintaining level control and constant feed flow to the fermentation tank, and negatively impacts fermentation yield. The fermentation process creates heat, which must be removed to continue effective fermentation, and the elevated entrained gas levels create heat removal issues in two ways. First, elevated entrained gas volumes generate cavitation in recirculation pumps that push the fermentation liquid through heat exchangers. This cavitation, and resulting flow loss, reduces the process capability to control the temperature. The entrained gas increases the thermal resistance of the bulk liquid to heat transfer which leads to a decrease in the heat exchange efficiency between the wort and the cooling water.

A system and method are needed to optimize the sugarcane fermentation process for the production of ethanol by monitoring and controlling the entrained gas content in the fermentation vessels used in the fermentation process.

Antifoam and/or defoamer chemistries have been developed for use in reducing foaming. In existing systems, antifoam chemistries are commonly added at three primary dosing points: at the top of the fermentation tank, in the treated yeast line, and in the sugarcane juice line entering the fermentation tank. Current practices rely in a continuous base loading of chemistry in the yeast and juice line and an intermittent slug dosing at the top of tank as a back-up system where the continuous dosing fails to adequately control foam volume, which can occur for various reasons. One such prior art back-up system is triggered primarily by conductance type probes installed at or near the top of the fermentation vessel(s). The rising foam reaches a critical level where the probe is installed, and touches the probe which triggers a slug of antifoam to be applied, often directly into the top of the tank. Thus, this type of system requires an upset to occur before the system can initiate, whereby the system is already in a state of inefficiency by the time the slug of antifoam is administered, necessarily resulting in production losses. Moreover, the slug is the final backup mechanism designed to control foam before it causes system failure or shutdown. Therefore, the slug is typically an excess dose of antifoam chemistry designed to control both nominal and severe system upset, and the result is that the maximum volume of antifoam is applied in each case, resulting in waste. There is currently no known means of adjusting the volume of this antifoam slug to account for the amount of excess foam in the system, let alone to proactively monitor foam level and adjust antifoam application in real time.

What is needed, then, is a system and method for actively monitoring foaming in fermentation vessels, and for proactively adjusting, in real time, the volume of antifoam chemistry (or other antifoam mechanisms) entering and/or acting on the fermentation vessel, in real time. It would be an added benefit if such a system actively monitored other process parameters which may impact foam levels, and recommended and/or implemented antifoam dosing levels based on a factoring of all relevant known parameters.

In addition, some existing ethanol processing facilities use two or more fermentation tanks, arranged in series, to conduct the fermentation process. In these facilities, the use of antifoam chemistry (including large, intermittent slugs of antifoam chemistry) in one or more of the upstream fermentation vessels may have a detrimental impact on the efficiency of the fermentation process downstream, and/or may eliminate the need for antifoam chemistry downstream. However, no system is known to account for the effects of anti-foam chemistry at other points along the processing line, and the same dose of antifoam chemistry is applied to downstream tanks irrespective of what is happening upstream.

Therefore it would be an even greater benefit to have a system which, where two or more fermentation vessels are operating in series, or where fermentation is conducted across multiple vessels in series or parallel, could centralize control of antifoam chemistry dosing based on real-time, measured parameters across the entire fermentation process line.

The problems caused by excessive entrained gas are compounded if the fermentation process doesn't result in a complete or nearly complete consumption of the sugars in the liquid. In that case, yeast will continue to consume the remaining sugars and generate CO₂ (and ethanol) further down the processing line (for continuous processes), which could cause additional efficiency losses in the process as a whole, throw off the calculation of how much antifoam chemistry to add further up the processing line, and/or cause unnecessary wear and tear on downstream equipment. Further, the failure of the fermentation process to completely remove sugars from the processing liquid represents an inefficient system and wasted materials.

Therefore, it would be especially advantageous if such a system was able to provide optimization parameters for the overall fermentation process.

### SUMMARY OF THE INVENTION

The present invention achieves these goals with a novel predictive control system for controlling foaming in fermentation vessel(s) while optimizing the fermentation process.

The invention comprises one or more gas volume fraction measurement devices operatively connected to one or more controllers and one or more deaeration mechanisms or other process regulating device which receive control signals from said one or more controllers and perform an act on the system, such as by controlling a level of deaeration chemistry or other inputs into some portion of the fermentation system.

In one embodiment, a deaeration mechanism according to the present invention is an antifoam feed pump which pumps antifoam chemistry into a feed line of the fermenter in response to the measured gas volume fraction in the fermenter's recirculation loop, in an amount determined by the controller to be effective to reduce foaming and lower column height in the fermenter. This predictive control system prevents the prior art problem of “over-dosing” the fermenter system with antifoam chemistry, or requiring a system upset in order to effectively control foaming.

In other preferred embodiments, the one or more gas volume fraction measurement devices are operatively connected to, in addition to or as an alternative to deaeration mechanisms, other process regulating devices which control the speed of the fermentation process in the system. In this way, measurements from the one or more GVF measurement devices can provide control signals useful in optimizing the fermentation process, resulting in a more complete fermentation.

The invention may be applied to large scale, batch or continuous fermentation operations by adding multiple GVF measurement devices along the processing line, which GVF measurement devices are monitored individually or centrally, and wherein a centralized controller may control deaeration devices across the entire processing line.

Additional embodiments of the present invention are envisioned wherein the inventive system is expanded by the addition of more measurement devices (measuring other processing parameters such as temperature, pH, flow rate, etc.) and other deaeration mechanisms, such as mechanical foam dispersant means, or other process regulating devices, such as pumps which control the flow rate of various processing lines or regulators which control the length of the fermentation process hold time.

The foregoing objects, features and attendant benefits of this invention will, in part, be pointed out with particularity and will become more readily appreciated as the same become better understood by reference to the following detailed description of a preferred embodiment and certain modifications thereof when taken in conjunction with the accompanying drawings.

### BRIEF DESCRIPTION OF THE DRAWINGS

In the drawings:

FIG. 1 is a process diagram showing a continuous fermentation operation involving a total of twelve fermentation tanks.

FIG. 2 is a process diagram of a simplified version of the fermentation process showing a single fermentation vessel and components of one preferred embodiment of the present invention.

FIG. 3 is a process diagram showing an exemplary installation of one embodiment of the disclosed invention for sugar ethanol fermentation

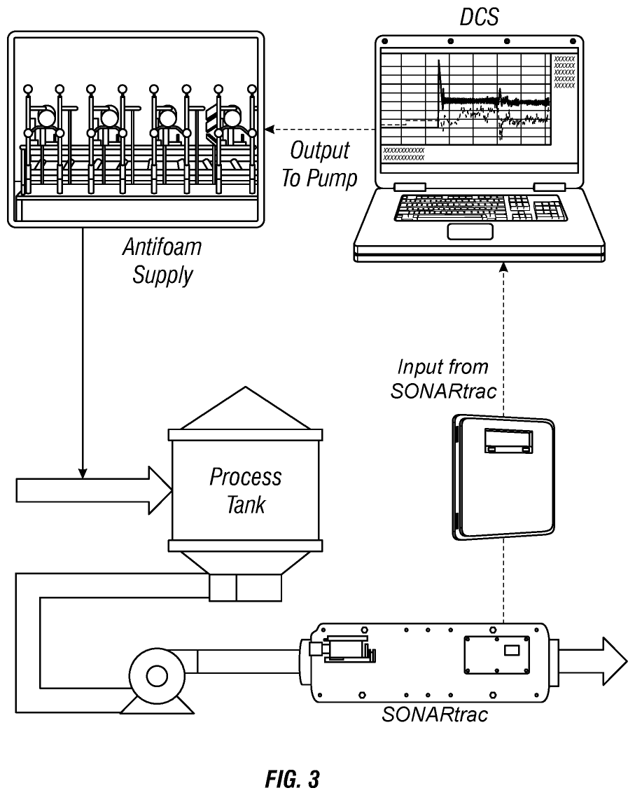

FIG. 4 is a process diagram showing an exemplary installation of one embodiment of the disclosed invention for sugar ethanol fermentation.

FIG. 5 is a graphical representation of a continuous 160-200 liters per minute flow through the GVF measurement devices in accordance with one embodiment of the present invention.

FIG. 6 is a comparison of the GVF data before and after the system of the present invention was enabled according to one embodiment.

FIG. 7 shows data obtained after enabling the automatic control according to one embodiment of the present invention.

FIG. 8 shows the architecture providing cloud connectivity for the inventive system.

FIG. 9 is a composite (A and B) of exemplary screen shots of a display unit comprising a mobile device running a mobile application programmed to provide the display.

### DETAILED DESCRIPTION OF THE PREFERRED EMBODIMENTS

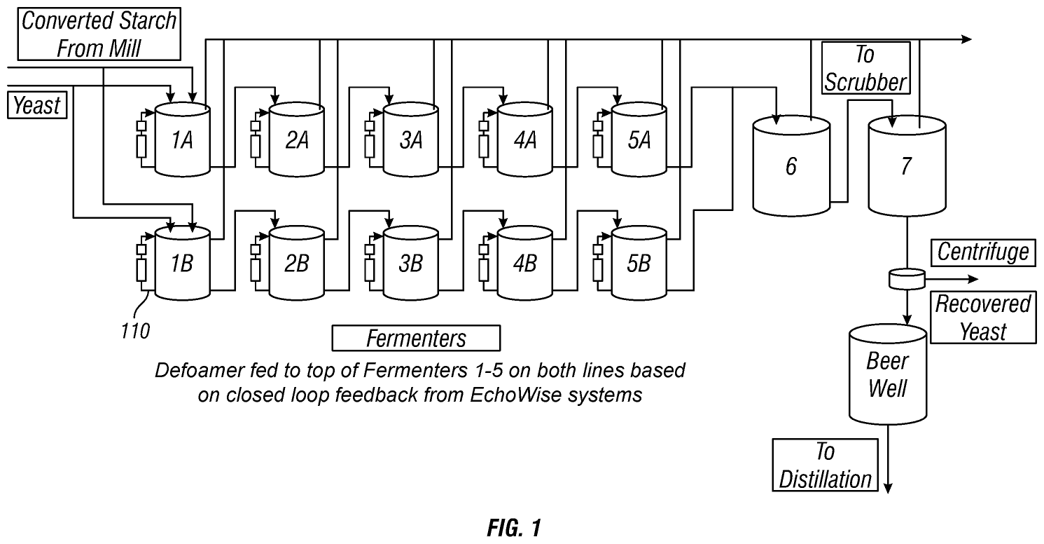

FIG. 1 is a process diagram showing a continuous fermentation operation involving a total of twelve fermentation tanks: ten tanks (1A-5A and 1B-5B) operating in two separate series parallel to each other, followed by two additional tanks (6 and 7) operating in series with the products of tanks 1A through 5B.

Regardless of the configuration of the fermentation tanks, in the conventional sugarcane fermentation process, the fermentation vessel (or initial fermentation vessel in a series) has two feed lines: (A) converted starch (such as sugarcane juice); and (B) yeast. The fermentation vessel also has a recirculation loop (designated as 110 with respect to vessel 1B in FIG. 1) which continuously draws liquid from the fermentation tank and passes it through a cooling loop (heat exchanger) in order to regulate the temperature of the material inside the fermentation tank. Output from the fermentation tank is fed into the next fermentation vessel in the series, or onto the next processing stage. This operation can be done continuously or in batches.

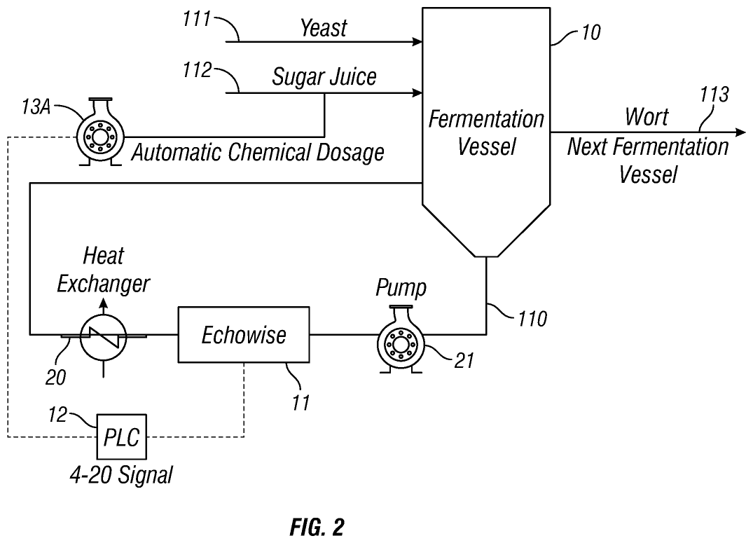

FIG. 2 is a process diagram of a simplified version of the fermentation process showing a single fermentation vessel and components of one preferred embodiment of the present invention. Yeast 111 and starch 112 are fed into the fermentation vessel 10. The recirculation loop 110, containing material from inside the fermenter, exits the bottom of the fermentation vessel (designated here as 10) and is pumped through a heat exchanger 20 to be cooled before returning to the fermentation vessel 10. The recirculation loop typically operates on a continuous basis. Note that, although a pump 21 is shown upstream from heat exchanger 20 in FIG. 2, the inventive system and method can be applied regardless of the configuration of the heat exchange loop. Wort 113 exits the fermentation vessel 10 and continues on to the next fermentation vessel in series or to further processing. This can be done in batches or as a continuous process.

In one preferred embodiment, the system of the present invention comprises: (A) at least one gas volume fraction (GVF) measurement device; (B) at least one controller; and (C) at least one deaeration mechanism. In another preferred embodiment, the system further comprises: (D) at least two GVF measurement devices; and (E) at least one process regulation device. Other components can be integrated into the system in preferred embodiments, as will be described, such as other measurement devices and other components of a control system that enable remote monitoring and control of the inventive system.

As used herein, the term “gas volume fraction (GVF) measurement device” means any such device known in the art or hereafter developed which is capable of determining the gas volume fraction, or quantity of gas, in a liquid or other medium, including gas that manifests in the form of bubbles or foam. Preferred embodiments of this invention utilize a sonar-based GVF measurement device, such as that disclosed in U.S. Pat. No. 8,109,127, the disclosure of which is incorporated by reference herein. Other potential GVF measurement devices which may be utilized according to the present invention are devices which utilize mass flow meters (such as the OPTIMASS Coriolis mass flowmeters sold by KROHNE Group) devices operating on principles of gamma-ray detection (such as the Roxar 2600 Multiphase Flow Meters sold by Emerson), devices which operate by measuring ultrasonic oscillation and/or ultrasonic intensity (such as that disclosed in Japanese Patent Application Publication No. 2002071647A), and other devices known in the art.

A deaeration mechanism according to the present invention could be one or more devices or treatments, including liquid, solid, or gaseous chemical compositions, known in the art to have the effect of reducing foam when applied to or in a mixture. Foaming/foam can generally be described as a gas bubble matrix entrained in, rising through, and/or resulting at the top of a liquid column. For example, a deaeration mechanism may include one or more liquid chemicals commonly referred to in the art as antifoam or defoamer chemistry (these being collectively referred to as “deaeration chemistry”). Examples of deaeration chemistry include silicone concentrates or emulsions, poly-alkalene glycol based, ester based, hydrophobic silica containing, and/or oil based (including mineral and vegetable) products, fatty alcohols, and other chemistries capable of de-aerating liquids and/or disrupting a foam matrix. A deaeration mechanism consisting of one or more antifoam or defoamer chemistries may be applied to the fermentation system by pumping them in liquid form into one or more feed lines of the fermenter or directly into the fermenter itself, as will be described.

A process regulation device according to the present invention could be a pump positioned to control the flow rate of various processing lines, regulators which control the length of the fermentation process hold time, or another device capable of controlling the overall length (in time) of the fermentation process. Process regulation device(s) could include: (A) one or more fermentation process pumps (that is, one or more wort pumps and/or one or more yeast pumps and/or one or more pumps delivering a combined flow of wort and yeast); (B) an automatic or manual adjustment valve between fermentation vessels. In the latter case, as the valves between vessels are opened the level in the previous vessel would tend to drop, and process supply pumps will speed up to return level to set point. Multiples of the above types of process regulation devices can also be used simultaneously, independently or dependently controlled, to produce the desired result.

Thus, with further reference to FIG. 2, one preferred embodiment of the invention includes a GVF measurement device, such as the ECHOWISE® sonar-based GVF measurement device marketed by Buckman Laboratories International, installed on one or more measurement locations associated with the fermentation tank. In FIG. 2, the GVF measurement device 11 is shown installed directly on recirculation line 110. In alternative embodiments, the GVF measurement device is installed in a slip stream configuration on the recirculation line 110, in an in-line or slip stream configuration on one or more input lines, 111, 112, and/or directly in the wall of the fermentation vessel 10, for GVF measurement devices that have such capability. Further, the GVF measurement device may be installed in any of the configurations described herein or otherwise known in the art on one or more of the lines carrying product between fermentation vessels, such as “wort” output line 113 or one or more feed lines. This invention is capable of being utilized with any device capable of measuring gas volume fraction, now known or developed in the future, and it will be understood that such device could be integrated with the inventive system in any configuration in which such device is designed to operate.

It will be understood that for systems or processing lines which incorporate multiple fermentation tanks, a GVF measurement device could be integrated with each such fermentation tank, its input or output feeds, and/or its recirculation line. In preferred embodiments, a GVF measurement device is integrated with each of the first and last vessels in series. In the case where two or more GVF devices are used in a particular system, they may be integrated with one another and/or centrally controlled as will be described herein.

Additional embodiments of the present invention include means for measuring other parameters of the fermentation operation, such as temperature, pH, mixing speed(s), residual sugar measurements, foam level, gas volume fraction on recirculation line, fermenter pH, inlet or outlet pH, fermenter level, residence time, sugar losses on fermentation, fermentation temperature, fermentation recirculation pressure, alcoholic degree, ethanol (or any other alcohol content), mash viscosity, yeast concentration, residual sugar measurements and/or flow rate of one or more processing, input and/or recirculation lines. The present invention is designed to incorporate means for measuring any parameter associated with the fermentation operation, and is not limited to one or more in particular (collectively referred to herein as “auxiliary measurement devices”). Such auxiliary measurement devices can be installed or incorporated in any and all configurations for which that particular device(s) was designed to be utilized with respect to any portion of the fermentation operation.

Regardless of the configuration of either GVF measurement device(s), the fermentation vessel(s) on which they are installed, or auxiliary measurement devices, in preferred embodiments of the present invention, each such measurement devices is operatively connected to a controller.

Also as used herein, the term “controller” may refer to any device capable of receiving input from the various measurement devices comprising the system according to one or more embodiments of the present invention, and processing that signal to transform it into a control signal of the type required by the deaeration mechanism or process regulation device utilized in each instance. By way of example only, the controller according to the present invention may be a programmable logic controller (PLC) which takes the signal received from the GVF measurement device and, based on variable programming, sends a control signal to the deaeration mechanism or process regulation device to cause that mechanism/device to act on the system in a manner and to a degree optimized to reduce foam in the fermentation vessel or change the processing speed, as may be the case.

In one embodiment, the controller includes a processor and memory sufficient to receive and record all available inputs from the various measurement devices described herein, and to provide output to one or more deaeration mechanisms, all in real time. Such a controller could modulate parameters of the deaeration mechanism(s) (such as dose rate of antifoam), while simultaneously measuring the gas fraction, flow rate, pH, temperature and other relevant parameters from all fermenters in the system to produce a response matrix. The system could then use the matrix to determine the optimal conditions that result in the highest fill level of fermenters to produce the maximum ethanol output. The response matrix can be set to adjust itself continuously to enhance the performance prediction. Where antifoam is one of the deaeration mechanisms used to reduce foam in the system, the controller could determine a lowest viable dosage of antifoam needed to maintain an acceptable level in the one or more fermenter vessel(s). Or the system could determine outputs for both lowest acceptable antifoam dosage (or other deaeration parameter), as well as outputs for optimum fermenter fill level, and calculate a weighted average for each output parameter based on the operator's goals for the system, e.g. to reduce antifoam dosage and/or increase efficiency.

The controller would then generate one or more control signals to the one or more deaeration mechanisms, respectively, to implement the controller-determined optimized levels for each such deaeration mechanism. Preferred embodiments of the system will perform this process continuously, in real time, to form a predictive control system for controlling foaming in the fermentation vessel(s). Such system may discover, with respect to a particular system's setup, that one or more measurable parameters (in addition to or as an alternative to GVF) is result-effective with respect to efficiency or other desired characteristic of the system, and may be able to proactively adjust one or more deaeration mechanism(s) to maintain such parameter(s) within an optimal range, thereby preventing system upset. Although each fermentation system may be different, one anticipated advantage to be obtained by the use of the inventive system is the reduction in volume of defoamer use and the resultant cost savings.

In fermentation systems which utilize more than one fermenter, the benefits of the disclosed system may be magnified by the use of multiple measurement devices (including multiple GVF measurement devices and/or multiple auxiliary measurement devices) across the system. For example, in some preferred embodiments, regardless of the overall configuration of the fermentation system (but with particular reference to systems which operate with several fermentation vessels in series), at least one GVF measurement device is installed at the front end of the fermentation operation (such as on the recirculation line of the first fermentation vessel in series) and at least one additional GVF measurement device is installed at or near the end of the fermentation process (such as on the recirculation line of the last fermentation vessel in series, or on the line exiting the last fermentation vessel headed for the next stage of processing). In addition to providing important operational data to the control system operating deaeration mechanism(s) in the system, such a configuration of GVF measurement devices will provide data on the change in entrained air between the beginning and end of the process, and will also capture important data on entrained gas quantity at or near the end of the fermentation process for use by the system in measuring completeness of the fermentation reaction. As described herein, larger amounts of entrained gas measured specifically at or near the end of the fermentation process could indicate that the fermentation reaction is incomplete, which may mean that the system is not operating at peak efficiency in that residual sugars remain at the end of the fermentation process, and in that the continuing consumption of those residual sugars by the yeast creates more CO₂ gas and compounds the entrained gas-caused inefficiencies in the overall process.

Therefore, in certain preferred embodiments, in addition to receiving measurements from GVF measurement devices located so as to provide optimal feedback for deaeration mechanisms, the system would receive GVF measurements from GVF measurement devices located at or near the beginning and end of the overall fermentation process (these may be the same or additional GVF measurement devices as already described with respect to a deaeration signal) and, optionally, from the results of any residual sugars testing done continuously or periodically in the system. All of the information described herein can be fed into the system's response matrix, and optimal levels for one or more deaeration mechanisms as well as the speed of the overall fermentation process can be determined to produce maximum efficiency in the system. Maximum efficiency can be measured and/or controlled by: (A) the lowest achievable residual sugar measurement at the end of the fermentation process; (B) highest speed of the overall fermentation process within a given foam level high set point; (C) optimum fermenter fill level; (D) lowest anti-foam chemistry dose level up to a given foam level high set point; (D) a combination of all of the above factors which taken together produce the highest ethanol output rate; or (E) some other control parameter at the operator's choosing. In preferred embodiments, action of one or more deaeration mechanism(s) and one or more process regulation device(s) can all be controlled in real time and in coordination with one another by a single system to produce optimal conditions based on the desired control factor(s).

For example, in certain embodiments one discrete input is the reading from a conductance probe in the head space of a fermentation vessel. A primary prior art foam control strategy is based on the detection of foam by a conductance probe in the vessel head space, which leads to the controller delivery a dose of liquid defoamer reagent. This is a back-up system where the continuous dosing of antifoam fails to adequately control foam formation. This defoamer dosage can be done via a pump (in most cases, a peristaltic pump is used) with a delay time ensure the defoamer reagent had adequate time to reduce the foam level before another shot of defoamer is added. So should the probe be activated, the pump doses a fixed rate of defoamer for a fixed time, so there is a timer to this control. Another common type of defoamer dosage apparatus is the one that uses a pneumatic cylinder with volume regulation of the defoamer shots. Again, the system is activated once the conductance probe detects foam, but in this case, product injection is made via the cylinder. In preferred embodiments, the inventive system integrates the monitoring and control of defoamer dosage via the described apparatus with the conductance probe.

Even greater benefits may be reaped by centralizing control of each such measurement device by installing them all in operative communication with a single (or comparatively lower number of) controller(s). The result is a predictive control system that would operate all of the interconnected measurement devices, process regulation device(s) and deaeration mechanism(s) as a whole. One possible benefit of such a system is the identification of redundant measurement devices, whereby the level of foam in the system could be adequately controlled using the remaining devices, thus providing a cost savings to the operator. This interconnected system could also lower demand for antifoam chemistry by applying it at the optimal point in the production process, e.g. when several fermenters are operated in series and antifoam chemistry will pass downstream through the processing line, lowering the downstream antifoam demand.

In preferred embodiments, a system according to the present invention includes a cloud computing system enabling remove visibility of GVF measurement units (and other measured system parameters as desired), and providing remotely visible standard dashboards for GVF measurement units or groups of units based on operator preference. The data insights generation is enabled by the development of a digital architecture, able to collect information from multiple sources, in order to store it in integrated databases and to make it available online. Technology also provides cloud based computational resources that allows the processing of large amounts of data using analytics tools, turning the collected data into real-time actionable information. By collecting real-time data and synthesizing it with data available online, the inventive system thus enables predictive control of the fermentation system for reduction/control of entrained gas volume and optimization of ethanol production.

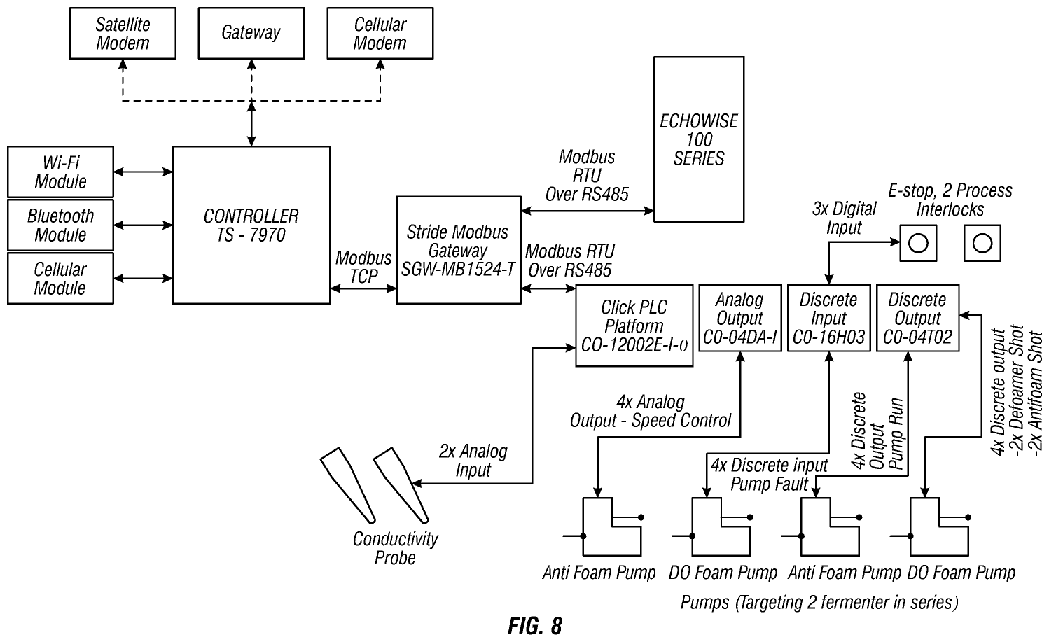

To integrate the digital and analog inputs and outputs and to connect the gas volume fraction measurement device via Modbus RTU, the solution created involves the use of a PLC as an TO rack and the integration of the gas volume fraction measurement device through separate hardware, more specifically, a ethernet serial Modbus gateway that supports four different serial connections. As to the cloud connectivity, the solution uses a modem or a gateway in order to send data to the cloud. This architecture is shown in FIG. 8.

More specifically, the inventive controller according to the present invention receives and records available inputs from various measurement devices and will aim to keep at targets or within ranges (one or more of) these fermentation process parameters (a/k/a Controlled Variables): foam level, gas volume fraction on recirculation line, fermenter pH, inlet or outlet pH, fermenter level, residence time, sugar losses on fermentation, fermentation temperature, fermentation recirculation pressure, alcoholic degree, ethanol (or any other alcohol content), mash viscosity, and/or yeast concentration. Such a controller preferably modulates the parameters of the deaeration mechanism(s) (such as dose rate of antifoam), while simultaneously measuring the controlled variables listed above to produce a response matrix. Preferred embodiments of the system then use the matrix to determine the optimal conditions that result in the highest fill level of fermenters to produce the maximum ethanol output. The response matrix can be set to adjust itself continuously to enhance the performance prediction. Where antifoam is one of the deaeration mechanisms used to reduce foam in the system, the controller could determine a lowest viable dosage of antifoam needed to maintain an acceptable level in the one or more fermenter vessel(s). In other preferred embodiments, the system determines outputs for both lowest acceptable antifoam dosage (or other deaeration parameter), as well as outputs for optimum fermenter fill level, and calculate a weighted average for each output parameter based on the operator's goals for the system, e.g. to reduce antifoam dosage and/or increase efficiency. In order to keep the controlled variables at target or within ranges, the controller will provide output to one or more deaeration mechanisms in real time and manipulate (one or more) of the following variables around the fermentation (a/k/a Manipulated Variables): antifoam flow, defoamer flow, inlet juice flow, yeast flow, yeast dilution flow, acid correction flow, lime correction flow, recirculation pump speed, and/or fermentation outlet flow.

The controller would then generate one or more control signals to the one or more deaeration mechanisms, respectively, to implement the controller-determined optimized levels for each such deaeration mechanism. In order to correlate any Manipulated Variable with any Controlled Variable and control the process, the controller can use one or more algorithms/strategies known in the art, including Directly Linear Correlation and Control (a straight line (y=ax+b) is used to determine what is the best value of the manipulated variable for each of controlled variables), piece-wise linear correlation (if the correlation between a manipulated variable and a controlled variable is not a straight line, the curve will be divided into a number of linear regions and an interpolation will be used between regions), Transfer Functions—Laplace Transforms (a function G(s) will be used to individually correlate each manipulated variable with each controlled variable. This function considers a specific gain between the manipulated variable and the controlled variable, as well as a delay (called dead time) between the end of the manipulated variable movement and the beginning of the controlled variable response), purely non-linear/phenomenological/equation-based control (the correlation between specific manipulated and controlled variables will be determined by an equation, which by nature is non linear. This equation can include any mass balance, energy balance or can combine both into a single system. These equations can be simple polynomial equations or differential ordinary equations and they can be used alone or organized into an equation system)

The inventive system uses one or more of the above mathematical strategies, or others known in the art for processing data of this type, separately or combined, in one of the following control scenarios: SISO (Single Input-Single Output) (one manipulated variable controlling one controlled variable only); MISO (Multiple Inputs-Single Outputs) (more than one manipulated variable controlling one controlled variable only); MIMO (Multiple Input-Multiple Output) (more than one manipulated variable controlling more than one controlled variable, organized into a “Controller Matrix”). Control signals for various components are generated by the system based on the control strategy or strategies utilized. The operator can, and/or the system can have pre-programmed, alarm threshold values for various parameters, whereby an alarm is triggered based on measurements meeting or exceeding the pre-set criteria, said alarm being visible or audible to the operator. Optional alarms can include: no signal from measurement device, no flow fermentation fluid flow on measurement device, measurement device power loss, pump fault, equipment loss of ethernet connection, low SOS quality, high GVF (such as GVF>10%), null GVF.

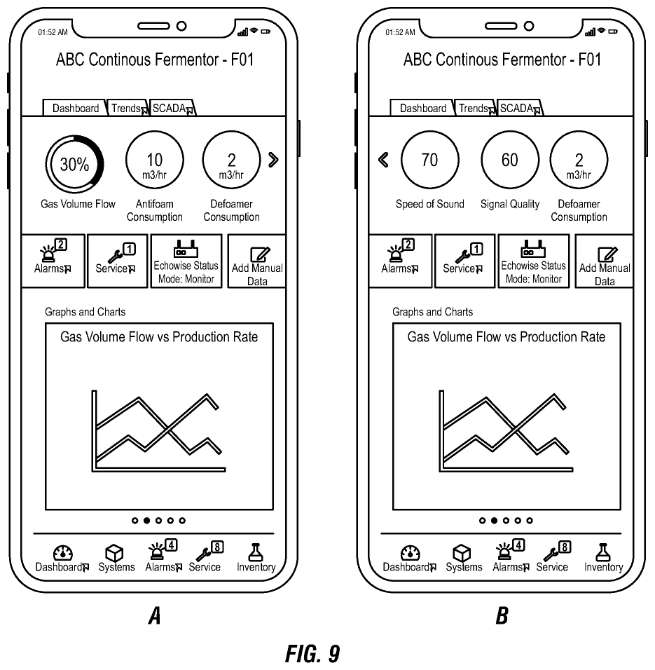

In preferred embodiments, the inventive system includes a display unit where the collected data, including Controlled Variables and Manipulated Variables, are all displayed in real time. The display unit can be remote from the processing line and the measurement devices, or located in the plant facility but connected to measurement and control devices via the cloud or other wireless network. The display unit preferably includes one or more dashboards that allow an operator to see metrics related to one or more GVF measurement devices or groups of devices, or generally related to one or more fermenters or groups of fermenters. The display unit can also display alarms in real-time as well as alarm history. In preferred embodiments, the dashboard is integrated with an IoT platform, performing cloud-based analytics in real-time, allowing 24/7 visibility of system operations and real-time tuning of operations through remote services. FIG. 9 shows exemplary screen shots of a display unit comprising a mobile device running a mobile application programmed to provide the display.

The inventive system also includes the integration of the solution with a digital platform that improves remote visibility and insights for end users, enables OTA (over the air) updates for the controller firmware, remote monitoring capabilities and the digitization of the entire application workflow.

In certain embodiments, the control and display software is downloadable to devices equipped with an Internet connection. The operator can then enter relevant information about the fermentation operation, as requested by the software, to set up the control system in connection with or following physical installation of GVF measurement units.

Again with reference to FIG. 2, a specific embodiment of an application of the inventive system to a single fermentation vessel to control dosage of antifoam chemistry is shown, although it will be understood that the same configuration described herein could be applied to one or more fermentation vessels in a system involving multiple such vessels in series and/or parallel.

In this embodiment, the antifoam feed pump 13A is configured to have a maximum dosage at 20 mA and a minimum dosage at 4 mA signal. The 4-20 mA signal of the pump input is converted by the controller 12 to a specific volume dosage. This closed-control loop will control the foam adequately and control the gas in liquid phase, adjusting the dosage as necessary by the process.

For a PLC 12 the pump 13A control signal can be calculated using the following equations; however, additional control strategies can be utilized, such as one or more of those described above.

$$GVF ( \% ) = ( \frac{EW DI}{4039} ) * EWOutRange$$

where:

EWOutRange is the 4-20 mA signal range configured at GVF measurement device, or in the case where the ECHOWISE® system is used, the ECHOWISE® transmitter;

EW DI is the output (in this case, analog, but devices capable of a digital output could be used with corresponding calculations) of the GVF measurement device (ECHOWISE® unit) in bits;

The number 4039 is the bit range of the digital to analog converter of the GVF measurement device (ECHOWISE® unit) (in case a 16-bit converter is used).

$$Output Pump Operation ( \% ) = ( \frac{PLC Output}{4039} ) * Factor 1$$

where:

Factor 1 is equal 100 to transform the output value in a percentage;

PLC Output is the digital output of the digital to analog converter of the controller;

The Output Pump Operation (OPO) is the percentage of maximum pumping rate of the pump 13A. The maximum pumping rate is determined experimentally to achieve a total foam abatement in a given application.

In embodiments where the deaeration mechanism is application of antifoam/defoamer chemistry, several possible dosing points are envisioned as compatible with the inventive system, including introducing antifoam into one or more input lines (in the case of sugarcane juice fermentation, into the sugarcane juice and/or yeast lines) and/or directly into the top of the fermentation tank.

Moreover, GVF measurement devices may be located in one or more positions relative to the fermentation tank(s), such as along one or more feed lines, recirculation lines, or in the wall of the fermentation vessel itself, all without departing from the scope of the present invention.

Although not specifically shown in FIG. 2, in certain preferred embodiments the same configuration of GVF measurement devices (or one of the other configurations described herein) is applied on both the first (or near first) and last (or near last) fermentation tank in a series. In this preferred embodiment, the controllers 12 affiliated with both GVF measurement devices are interconnected to a larger series of controllers and/or provides a wired (or wireless) signal to an overall system control substation (described in greater detail above). In preferred embodiments, one lead controller receives signals from each of the GVF measurement devices, and sends control signals not only to the deaeration mechanism associated with each individual fermentation vessel but also to one or more process regulation device(s) which can speed up or slow down the speed of the overall fermentation process, according to a control matrix described elsewhere herein (or based on manual inputs from an operator in receipt of all such collected data). The system could then, for example, speed up the rate of the fermentation process, and thereby increase production, where low GVF (signaling complete or near complete consumption of sugars by the process) measurements are obtained near the end of the processing line. Alternatively, the system could slow down the rate of the fermentation process where high GVF (signaling incomplete consumption of sugars [high residual sugars] by the process) measurements are obtained near the end of the processing line. In connection with either scenario, the system could then adjust conditions at the one or more deaeration mechanism(s) in accordance with the system's determination of optimal conditions for such device(s) based on the then-operative production rate, in real time.

### EXAMPLE

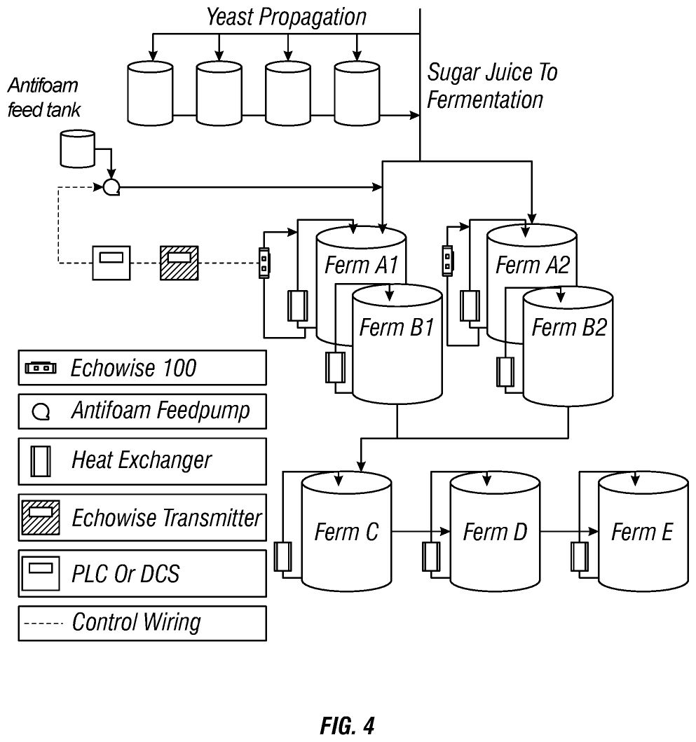

The method and system of the present invention was installed at a sugar mill in Brazil. The setup utilized, as GVF measurement devices, two ECHOWISE® units model TAM-100. With reference to FIG. 4, one unit was installed in a slip stream a first recirculation line and the other one in a slip stream on a second recirculation line, both recirculation lines being on the primary fermenters. The recirculation lines are used to control the temperature in the fermentation tank that increase with the exothermic fermentation biological process. The critical temperature for the process in tanks is 35° C./95° F.

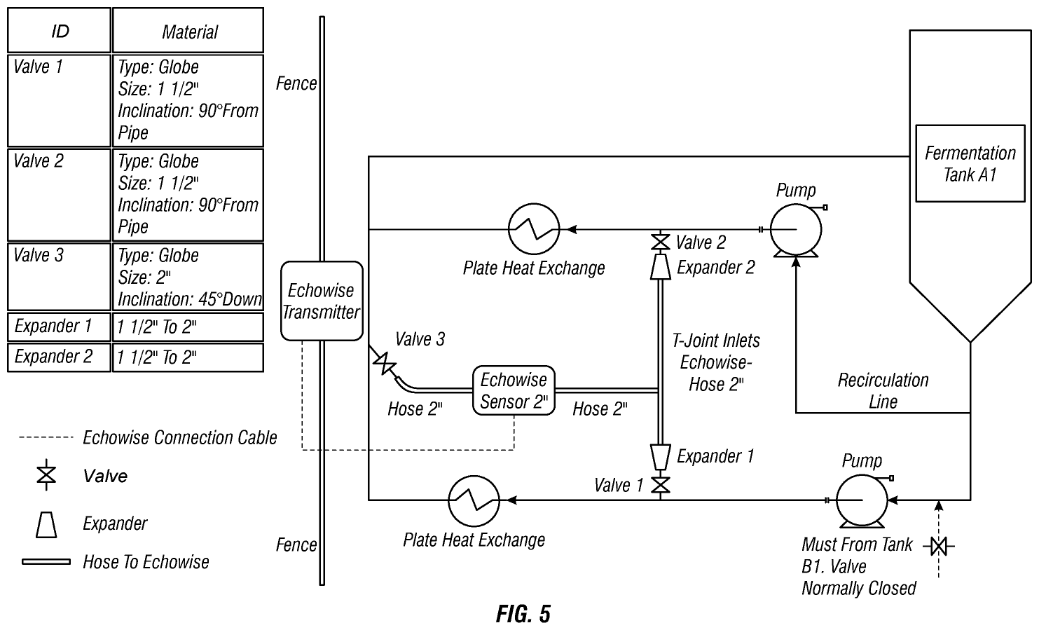

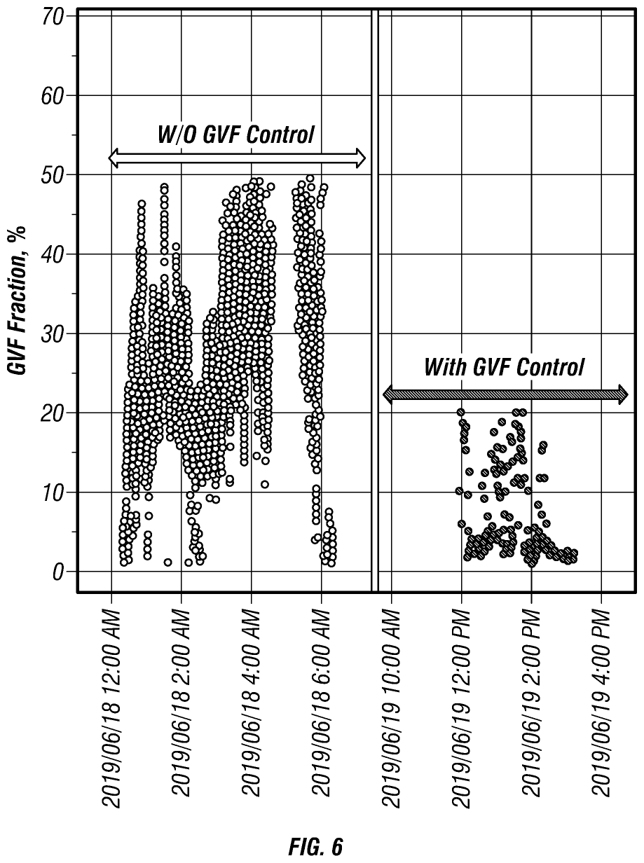

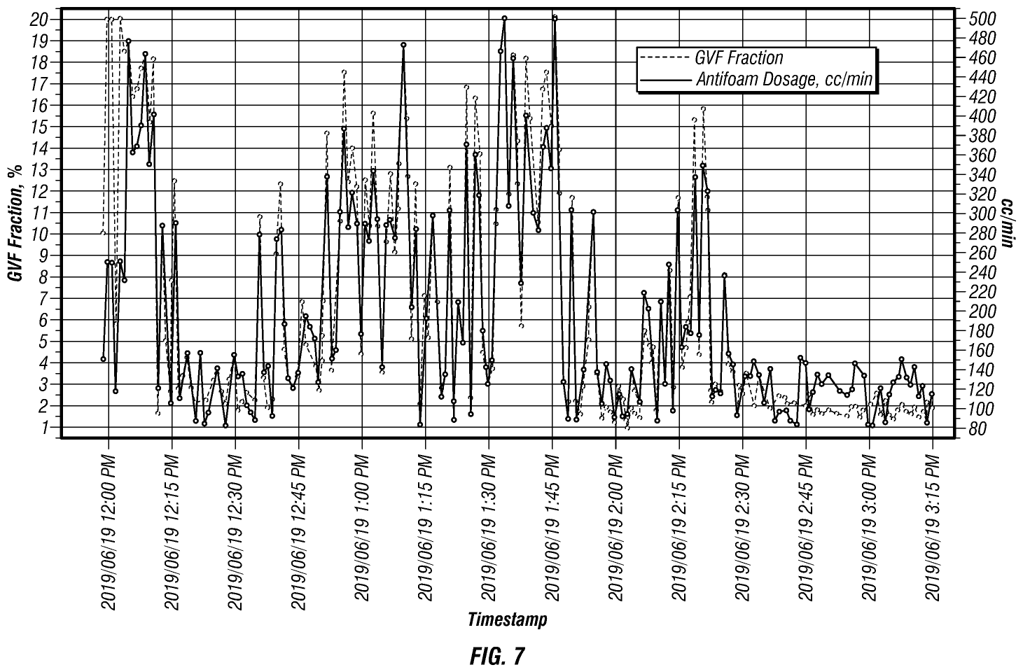

The inlet and outlet valves of the ECHOWISE® units were set up and adjusted to provide a continuous 160-200 liters per minute flow in accordance with the units' specifications as shown in FIG. 5. The comparison of the GVF fraction data obtained before and after enabling the automatic control of anti-foam feed is provided in FIG. 6. A significant decrease in the amount of entrained gas takes place as a result of the control. The data obtained after enabling the automatic control is provided in FIG. 7. Graphical representation of this data shows that there is still significant variability in GVF, but this variability is closely followed by the adjustments of the antifoam dosage. As a result of the method and system applied, the sugar mill was able to obtain a better flow and fermenter level stability on the fermentation line on which it was installed.

As can be seen, the above-described system, in its various embodiments applicable to fermentation operations of all scales and configurations, provides a comprehensive fermentation management system which beneficially reduces foaming and improves efficiency in fermentation operations, and particularly bio-ethanol production fermentation processes. Demonstrated benefits of the inventive system include: lower and more stable levels in fermentation vessels; ethanol production increases (in one field test, the system and method improved production from 125 m³/hr to 175 m³/hr); and reduction of additive use, including the reduction or elimination (in one field study) of the secondary dosing of defoamer, based on the conductance probe system, commonly used in prior art systems. Other potential benefits of the disclosed system may include other additive dosing reductions, including a possible reduction in the need for antibiotic dosing.

Yet additional possible uses or benefits of the inventive system include: reduction of total foam control chemistry (by optimization of the total foam control chemistry dosage); reduction in contamination in the fermentation operation (i.e., by a decrease in the microbiological contamination outbreaks observed in the fermentation, which in turn would likely increase the fermentation efficiency, decrease sugar losses cause by the competitions between bacteria and yeast and decrease the consumption of biocide used to control contamination); increase in fermentation efficacy (process optimizations and decrease in sugar losses are translated into an optimal conversion of fermentable sugars in ethanol, meaning a higher fermentation efficiency); reduction in sugar losses (foam formation is one of the variables that contribute to sugar losses in the fermentation process, and the system described here addresses the foam formation and the overall control of the fermentation process, which is translated into to reduction in sugar losses); and increase in process stability via integration of data from the gas volume fraction measurement devices (which, combined with process data and lab analysis can help mills to gain the necessary visibility to predict issues in the fermentation process and data driven decisions, increasing process stability).

While the device disclosed herein is particularly useful for use in biofuel fermentation operations, it is within the scope of the invention disclosed herein to adapt the device to use in other fields, and to fermenters or processing vessels of other types.

This application is therefore intended to cover any variations, uses, or adaptations of the invention using its general principles. Further, this application is intended to cover such departures from the present disclosure as come within known or customary practice in the art to which this invention pertains.

We claim:

1. A fermenter control system, the system comprising:
a gas volume fraction (GVF) measurement device;
a controller operatively connected to said GVF measurement device; and
one or more deaeration mechanisms operatively connected to said controller.

2. The fermenter control system of claim 1, wherein one of said one or more deaeration mechanisms is a mechanical foam control device.

3. The fermenter control system of claim 2, wherein one of said one or more deaeration mechanisms is a vacuum-based foam control device.

4. The fermenter control system of claim 1, wherein one of said one or more deaeration mechanisms is a first pump, wherein said first pump controls a flow rate of deaeration chemistry into a first processing stream.

5. The fermenter control system of claim 4, wherein said first processing stream is a feed of yeast into said fermenter.

6. The fermenter control system of claim 4, wherein said first processing stream is a feed of sugarcane juice into said fermenter.

7. The fermenter control system of claim 4, wherein said first processing stream is a feed of deaeration chemistry into the top of said fermenter.

8. The fermenter system of claim 1, wherein one of said one or more deaeration mechanisms is a second deaeration mechanism operatively connected to said controller.

9. The fermenter system of claim 8, wherein said fermenter system comprises at least two fermentation vessels in series, and wherein said first deaeration mechanism and said second deaeration mechanism each act on one of said at least two fermentation vessels.

10. The fermenter system of claim 9, wherein
said first deaeration mechanism is a pump which controls the feed rate of deaeration chemistry into a feed line of a first of said at least two fermentation vessels in series, and wherein
said second deaeration mechanism is a pump which controls the feed rate of deaeration chemistry into a feed line of a second of said at least two fermentation vessels in series.

11. The fermenter control system of claim 1, wherein said GVF measurement device is installed directly on the heat exchange unit loop of said fermenter.

12. The fermenter control system of claim 1, wherein said GVF measurement device is installed in a slip stream configuration around a heat exchange unit loop of said fermenter.

13. The fermenter control system of claim 1, wherein said GVF measurement device is installed directly on a feed line of said fermenter.

14. The fermenter control system of claim 1, wherein said GVF measurement device is installed on a first fermentation vessel in a series of fermentation vessels, and further comprosing a second GVF measurement device installed on a last fermentation vessel in a series of fermentation vessels.

15. The fermenter control system of claim 1, wherein said GVF measurement device is installed in a slip stream configuration around a feed line of said fermenter.

16. The fermenter control system of claim 1, wherein said GVF measurement device is installed in the wall of said fermenter vessel.

17. The fermenter control system of claim 4, further comprising:
a second pump operatively connected to said controller, wherein said second pump controls a flow rate of deaeration chemistry into a second processing stream.

18. The fermenter control system of claim 1, wherein said controller is a programmable logic controller comprising software configured to determine an appropriate amount of anti-foam chemistry based on inputs received from said GVF measurement device.

19. The fermenter control system of claim 1, wherein said controller is selected from a group comprising a direct analog or digital signal from a transmitter of said GVF measurement device or a variable frequency device such as a variable speed drive.

20. The fermenter control system of claim 1, further comprising one or more auxiliary measurement devices operatively connected to said controller, wherein said controller produces a control signal to said first deaeration mechanism based on inputs from said GVF measurement device and said one or more auxiliary measurement devices.

21. The fermenter control system of claim 20, wherein said one or more auxiliary measurement devices are selected from the list comprising temperature sensor, pH sensor, mixing speed sensor, and/or flow rate sensor for one or more processing, input and/or recirculation lines of said fermenter.

22. The fermenter control system of claim 20, wherein said controller comprises software configured to develop a control matrix to determine an appropriate target or target range for each of one or more Controlled Variables based on inputs received from said GVF measurement device and said one or more auxiliary measurement devices.

23. The fermenter control system of claim 22, wherein said one or more Controlled Variables are selected from a group comprising: foam level, gas volume fraction on recirculation line, fermenter pH, inlet or outlet pH, fermenter level, residence time, sugar losses on fermentation, fermentation temperature, fermentation recirculation pressure, alcoholic degree, ethanol (or any other alcohol content), mash viscosity, and/or yeast concentration.

24. The fermenter control system of claim 22, wherein the controller provides control signals to said one or more deaeration mechanisms, which control signals are designed to maintain said appropriate target or target range for each of one or more Controlled Variables.

25. The fermenter control system of claim 24, wherein said control signals are designed to control one or more Manipulated Variables for said one or more deaeration mechanisms, said Manipulated Variables being selected from a list comprising antifoam flow, defoamer flow, inlet juice flow, yeast flow, yeast dilution flow, acid correction flow, lime correction flow, recirculation pump speed, and/or fermentation outlet flow.

26. The fermenter control system of claim 24, wherein said controller is programmed to provide one or more audio or visual alarms in response to a measured deviation from said appropriate target or target range for each of one or more Controlled Variables.

27. The fermenter control system of claim 22, wherein said control matrix is programmed to determine optimal conditions that result in the highest fill level of fermenters to produce the maximum ethanol output.

28. The fermenter control system of claim 20, wherein said controller is operatively connected to a remote display system, said remote display system including means to display various parameters associated with said GVF measurement device and said one or more auxiliary measurement devices.

29. The fermenter control system of claim 1, wherein said controller is operatively connected to a remote display system, said remote display system including means to display various parameters associated with said GVF measurement device.

30. The fermenter control system of claim 1, further comprising a first process regulation device operatively connected to said controller.

31. A method of controlling liquid column height in a fermenter, the method comprising:
measuring a volume of entrained gas in a processing stream of said fermenter;
determining, based on said volume of entrained gas, operation parameters of one or more deaeration mechanisms optimized to control said liquid column height to below a predetermined level;
transmitting a control signal to said one or more deaeration mechanisms to implement said operation parameters.

32. The method of claim 31, wherein said volume of entrained gas is measured by a sonar-based measurement device.

33. The method of claim 31, wherein said deaeration mechanisms is a pump which controls addition of deaeration chemistry to a feed line into said fermenter in response to said control signal.

34. The method of claim 33, wherein said feed line is a feed of sugarcane juice into said fermenter.

35. The method of claim 33, wherein said feed line is a feed of yeast into said fermenter.

36. The method of claim 31, wherein said measuring step comprises measuring said volume of entrained gas in a heat exchange unit loop of said fermenter.

37. The method of claim 31, wherein said measuring step comprises measuring said volume of entrained gas in a feed line of said fermenter.

38. The method of claim 31, wherein said measuring step comprises measuring said volume of entrained gas inside said fermenter vessel.

39. The method of claim 31, further comprising the step of:
measuring one or more auxiliary parameters related to said fermenter, said one or more auxiliary parameters being selected from the group comprising temperature, pH, mixing speed and/or flow rate; and
wherein said determining step comprises determining, based on said volume of entrained gas and said one or more auxiliary parameters, operation parameters of one or more deaeration mechanisms optimized to control said liquid column height to below a predetermined level

40. The method of claim 31, further comprising:
determining, based on said volume of entrained gas, operation parameters of one or more process regulation devices optimized to control a processing speed of a fermentation reaction in said fermenter;
transmitting a control signal to said one or more process regulation devices to implement said operation parameters.

41. A method of reducing additive consumption in a fermenter, the method comprising:
measuring a volume of entrained gas in a processing stream of said fermenter;
determining, based on said volume of entrained gas, a flow rate of deaeration chemistry optimized to control said liquid column height to below a predetermined level;
transmitting a control signal to a pump to implement said flow rate of deaeration chemistry.
